(19) United States
(12) Patent Application Publication
(10) Pub. No.: US 2022/0395440 A1
(43) Pub. Date: Dec. 15, 2022

(54) SUN CARE COMPOSITIONS WITH HOLLOW MESOPOROUS SILICA NANOSPHERES

(71) Applicants: DOW GLOBAL TECHNOLOGIES LLC, Midland, MI (US); ROHM AND HAAS COMPANY, Collegeville, PA (US)

(72) Inventors: Jinxiu WANG, Shanghai (CN); Shiling ZHANG, Shanghai (CN); XiaoYi PANG, Shanghai (CN); Hongyu CHEN, Shanghai (CN); Fanwen ZENG, Audubon, PA (US)

(73) Assignees: DOW GLOBAL TECHNOLOGIES LLC, Midland, MI (US); ROHM AND HAAS COMPANY, Collegeville, PA (US)

(21) Appl. No.: 17/772,897

(22) PCT Filed: Oct. 30, 2019
(86) PCT No.: PCT/CN2019/114176

§ 371 Date: Apr. 28, 2022

(51) Int. Cl. A61K 8/25; A61K 8/37; A61K 8/02; A61Q 17/04

(52) U.S. Cl. CPC A61K 8/25; A61K 8/37; A61K 8/025; A61Q 17/04; A61K 2800/413

(57) ABSTRACT

Described herein are sun care compositions comprising at least one sunscreen active and hollow mesoporous silica nanospheres, and methods of making and using the sun care compositions. The presently described sun care compositions further comprise at least one of a cosmetically acceptable emollient, humectant, vitamin, moisturizer, conditioner, oil, silicone, suspending agent, surfactant, emulsifier, preservative, rheology modifier, pH adjustor, reducing agent, anti-oxidant, and/or foaming or de-foaming agent.

### BACKGROUND

Sun care compositions are typically personal care compositions designed to prevent a percentage of ultraviolet (UV) radiation coming from the sun from reaching the wearer's skin. UVA radiation (315 nm-400 nm) does not cause visible radiation burns (e.g., sunburn), but has been shown to cause indirect DNA damage through free radical generation. UVB radiation (290 nm-315 nm) causes sunburn in the short term, and is additionally associated with cancers (e.g., melanomas) over time.

Sunscreen actives, such as physical UV blockers (e.g., titanium dioxide, zinc oxide) and chemical UV absorbers (e.g., para-aminobenzoic acid, octyl methoxycinnamate), can protect a user from UVA radiation and/or UVB radiation. Sun protection factor (SPF) ratings are relevant to UVB blocking, and in theory, the higher the amount of sunscreen actives (such as, for example, UV filters), the greater the degree of the UV protection. However, too high a concentration of sunscreen active results in impairment of the composition's aesthetics (such as tackiness, greasiness, grittiness, whiteness, etc.) and/or undesirable toxicological effects. Consequently, finding ways to increase the SPF without adding more sunscreen actives, such as, for example, by finding synergistic combinations or by adding compounds which are not recognized sunscreen actives, but work to increase the SPF (referred to herein as SPF boosters), is an important goal in the personal care industry.

Accordingly, there is a need to identify SPF boosters which will help achieve higher SPF without increasing the concentration of sunscreen active.

### SUMMARY

Described herein are sun care compositions comprising at least one sunscreen active and hollow mesoporous silica nanospheres, and methods of making and using the sun care compositions. The presently described sun care compositions further comprise at least one of a cosmetically acceptable emollient, humectant, vitamin, moisturizer, conditioner, oil, silicone, suspending agent, surfactant, emulsifier, preservative, rheology modifier, pH adjustor, reducing agent, anti-oxidant, and/or foaming or de-foaming agent.

### BRIEF DESCRIPTION OF THE DRAWINGS

FIG. 1 is a Scanning Electron Microscope (SEM) image of a first group of hollow mesoporous silica nanospheres (HMSN-1).

FIG. 2 is a Transmission Electron Microscope (TEM) image of HMSN-1.

FIG. 3 is a TEM image of a hollow mesoporous silica nanosphere from a second group of hollow mesoporous silica nanospheres (HMSN-2).

FIG. 4 is a plot of nitrogen sorption isotherms for HMSN-1.

FIG. 5 is a plot of pore size distribution for HMSN-1.

FIG. 6 is a plot of nitrogen sorption isotherms for HMSN-2.

FIG. 7 is a plot of pore size distribution for HMSN-2.

FIG. 8A & FIG. 8B are diagrams of sun protection factor (SPF) measurements for comparative sun care formulations, sun care formulations including HMSN-1, and a sun care formulation including HMSN-2.

### DETAILED DESCRIPTION

Described herein are sun care compositions. A sun care composition is a personal care composition for protecting a user from UV radiation. Examples of sun care compositions include compositions having an SPF rating (for example, sunscreen compositions) and/or personal care compositions where a UV blocker would be beneficial, such as, for example, moisturizers, lip balms, etc.

The presently described sun care compositions contain one or more (e.g., mixtures) sunscreen actives. Sunscreen actives is intended to include physical UV blockers (e.g., titanium dioxide, zinc oxide) and chemical UV absorbers (e.g., para-aminobenzoic acid, octyl methoxycinnamate). Examples of suitable sunscreen actives include titanium dioxide, zinc oxide, para-aminobenzoic acid, octyl methoxycinnamate, ethylhexyl methoxycinnamate, ethylhexyl salicylate, Octocrylene (2-ethylhexyl-2-cyano-3,3 diphenylacrylate), butyl methoxydibenzoylmethane, Avobenzone (4-t-butyl-4′-methoxydibenzoyl-methane), oxybenzone, dioxybenzone, cinoxate (2-ethoxyethyl-p-methoxy-cinnamate), diethanolamine-p-methoxycinnamate, ethylhexyl-p-methoxy-cinnamate, isopentenyl-4-methoxycinnamate, 2-ethylhexyl salicylate, digalloyl trioleate ethyl 4-bis(hydroxypropyl)aminobenzoate, glyceryl aminobenzoate, methyl anthranilate, homosalate (3,3,5-trimethylcyclohexyl salicylate), triethanolamine salicylate, 2-phenyl-benzimidazole-5-sulfonic acid, sulisobenzone (2-hydroxy-4-methoxy-benzophenone-5-sulfonic acid), Padimate A (amyl p-dimethylaminobenzoate), Padimate 0 (octyl dimethyl para aminobenzoate), 4-Methylbenzylidene camphor, sunscreen actives sold under the tradenames ECAMSULE™, TINOSORB™, NEO HELIOPAN™, MEXORYL™, BENZOPHENONE™, UVINUL™, UVASORB™, and/or PARSOL™, and/or mixtures thereof. Preferably, the sunscreen active is a mixture of ethylhexyl methoxycinnamate, ethylhexyl salicylate, and butyl methoxydibenzoylmethane. Preferably, the sunscreen active is a mixture of ethylhexyl methoxycinnamate, ethylhexyl salicylate, butyl methoxydibenzoylmethane, and 2-ethylhexyl-2-cyano-3,3 diphenylacrylate (e.g., octocrylene).

Preferably, the present sun care compositions contain greater than about 10%, greater than about 12%, greater than or equal to about 13%, and less than about 20%, less than about 19%, and less than or equal to about 18%, total sunscreen active(s) by weight of the composition.

The presently described sun care compositions further comprise inorganic hollow mesoporous silica nanospheres (also referred to herein as HMSN). Hollow mesoporous silica nanospheres refer to nano-sized generally spherical silicon oxide particles comprising a shell defining a hollow interior portion. A plurality of pores (e.g., channels) pass through the shell, extending from the hollow portion to the exterior surface of the shell. As used herein, “mesoporous” refers to having pores with diameters from about 2 nm to about 50 nm.

Hollow mesoporous silica nanospheres are typically prepared by growing silicon oxide (e.g., using silicate precursors, such as, for example, alkoxy silanes, alkyl silicates, etc.) in the presence of one or more surfactants (e.g., ionic, nonionic, polymeric, organic, etc.) and, optionally, a spherical template compound, and subsequently removing the surfactant (e.g., and if present, the spherical template compound), for example, with acid (e.g., hydrochloric acid), to afford the hollow mesoporous silica nanospheres. Preferably, a tetra-alkyl silicate is combined with a surfactant mixture (e.g., comprising a nonionic triblock copolymer surfactant and an ionic surfactant containing an organic alkyl chain) in an alkyl alcohol and water. The reaction may be acid or base catalyzed. Hollow mesoporous silica nanospheres are commercially available, for example, from Shanghai Fuyuan Nano Mesoporous Materials Co. under the tradename LKHS-65.

Preferably, the presently described hollow mesoporous silica nanospheres have a particle size greater than about 150 nm, greater than about 200 nm, greater than or equal to about 250 nm, and less than about 450 nm, less than about 400 nm, and less than or equal to about 350 nm. Preferably, the presently described hollow mesoporous silica nanospheres have a particle size from between about 150 nm and about 400 nm. Transmission Electron Microscope (TEM) images may be used to determine particle size, measuring manually using a scale bar.

Preferably, the presently described hollow mesoporous silica nanospheres have a surface area greater than about 500 m²/g, greater than about 600 m²/g, greater than or equal to about 620 m²/g nm, and less than about 1200 m²/g, less than about 1100 m²/g, and less than or equal to about 960 m²/g. Preferably, the presently described hollow mesoporous silica nanospheres have a surface area of between about 600 m²/g and 1200 m²/g. Surface area is determined by the Brunauer-Emmett-Teller (BET) nitrogen gas adsorption method (e.g., Brunauer, S. et al., Adsorption of Gases in Multimolecular Layers, Journal of the American Chemical Society, pp. 309-319 (1938) is incorporated by reference herein in its entirety). Specific surface areas of porous materials may be calculated by Equation (1):

$$\frac{ρ}{v ( {ρ}_{0} - ρ )} = \frac{1}{{v}_{m} c} + \frac{c - 1}{{v}_{m} c} \frac{ρ}{{ρ}_{0}} \qquad ( 1 )$$

where v is the adsorbed volume of gas, vₘ is the monolayer saturation absorption volume, p is the equilibrium gas pressure, p₀ is the saturation pressure, and c is the BET constant. The y-intercept and slope of this function can then be used to solve for the constants c (=slope/intercept+1) and vm (=1/(slope+intercept). The specific surface area (S, surface area per unit mass) can then be found by Equation (2):

$$S = {A}_{x s} ( \frac{{v}_{m} N}{22 , 414} ) \qquad ( 2 )$$

where N is Avogadro's number, Aₓₛ is the cross-sectional surface area of a single adsorbed gas molecule, and 22,414 represents the standard temperature and pressure (STP) volume of one mole of gas.

Preferably, the presently described hollow mesoporous silica nanospheres have a shell thickness greater than about 10 nm, greater than about 20 nm, greater than or equal to about 25 nm, and less than about 100 nm, less than about 80 nm, and less than or equal to about 60 nm. Preferably, the presently described hollow mesoporous silica nanospheres have a shell thickness of between about 10 nm and 100 nm. Transmission Electron Microscope images may be used to determine shell thickness, measuring manually using a scale bar.

Preferably, the presently described hollow mesoporous silica nanospheres have a generally spherical hollow cavity with a diameter greater than about 100 nm, greater than about 150 nm, greater than or equal to about 200 nm, and less than about 300 nm, less than about 275 nm, and less than or equal to about 250 nm. Preferably, the presently described hollow mesoporous silica nanospheres have a hollow cavity diameter of between about 100 nm and 300 nm. Transmission Electron Microscope images may be used to determine cavity diameter, measuring manually using a scale bar.

Preferably, the presently described hollow mesoporous silica nanospheres have a pore size greater than about 1 nm, greater than about 2 nm, greater than or equal to about 2.2 nm, and less than about 4 nm, less than about 3 nm, and less than or equal to about 2.6 nm. Preferably, the presently described hollow mesoporous silica nanospheres have a pore size of between about 2.0 nm and 4.0 nm. Pore size distributions are determined using the Barrett-Joyner-Halenda (BJH) model (e.g., Barret, E. et al., Determination of Pore Volume and Area Distributions in Porous Substances. I. Computations from Nitrogen Isotherms, Journal of the American Chemical Society, pp. 373-380, vol. 73 (1951) is incorporated by reference herein in its entirety). Pore sizes are derived from the adsorption branches of isotherms, using Equation (3):

r_{P}=rₖ+t (3)

where rₚ is the radius of a pore, rₖ is “Kelvin radius,” computed from the following classical Kelvin equation (Equation (4) below), and t is the adsorbed multilayer thickness. Values oft as a function of the relative pressure are obtained from a plot of the experimental data. Equation (4) is as follows:

$$log ( P / {P}_{0} ) = \frac{- 2 σ V}{8.316 \times {10}^{7} \times 2.303 T {r}_{k}} = \frac{- 4.14}{{r}_{k}} \qquad ( 4 )$$

where σ is the surface tension of liquid nitrogen, V is the liquid molar volume of nitrogen, rₖ is the radius of the pore, T is the absolute temperature in Kelvin, and 8.316×10⁷ is the gas constant in ergs per degree.

Preferably, the presently described hollow mesoporous silica nanospheres have a particle size between about 250 nm and about 300 nm, a surface area of about 960 m²/g, a shell thickness of about 25 nm, a hollow cavity diameter of about 200 nm, and a pore size of about 2.4 nm.

Preferably, the presently described hollow mesoporous silica nanospheres have a particle size between about 350 nm and about 400 nm, a surface area of about 620 m²/g, a shell thickness of about 60 nm, a hollow cavity diameter of about 250 nm, and a pore size of about 2.2 nm.

Preferably, the present sun care compositions contain greater than about 2%, greater than about 3%, greater than about 4%, and less than about 7%, less than about 6%, and less than or equal to about 5%, hollow mesoporous silica nanospheres by weight of the composition. Preferably, the present sun care compositions contain about 3.3% hollow mesoporous silica nanospheres by weight of the composition. Preferably, the present sun care compositions contain about 5% hollow mesoporous silica nanospheres by weight of the composition.

Preferably, the present sun care compositions may comprise at least one of a cosmetically acceptable emollient, humectant, vitamin, moisturizer, conditioner, oil, silicone, suspending agent, opacifier/pearlizer, surfactant, emulsifier, preservative, rheology modifier, colorant, pH adjustor, propellant, reducing agent, anti-oxidant, fragrance, foaming or de-foaming agent, tanning agent, insect repellant, and/or biocide. Preferably, a sun care composition may contain at least one of a humectant, a surfactant, and/or an emollient.

In use, sun care compositions including the presently described hollow mesoporous silica nanospheres may be used to protect a mammal from damage caused by UV radiation (e.g., UVA radiation and/or UVB radiation). For example, a method of protecting a mammal (e.g., the skin of a mammal) from damage caused by UV radiation comprises applying the presently described sun care compositions to the skin of the mammal.

Preferably, the presently described hollow mesoporous silica nanospheres act as a sun protection factor (SPF) booster for the sun care compositions. Preferably, the SPF of the sun care composition is more than 25% higher than a comparative composition without the hollow mesoporous silica nanospheres.

The following examples are for illustrative purposes only and are not intended to limit the scope of the appended claims.

### EXAMPLES

### Example 1

Inorganic hollow mesoporous silica nanospheres are characterized as follows. Two kinds of hollow mesoporous silica nanospheres with variable particle sizes, cavity sizes (e.g., diameters), shell thicknesses, and porosities have been denoted as Hollow Mesoporous Silica Nanosphere Batch 1 (HMSN-1) and Hollow Mesoporous Silica Nanosphere Batch 2 (HMSN-2). Both HMSN products are white powders and display regular spherical morphology.

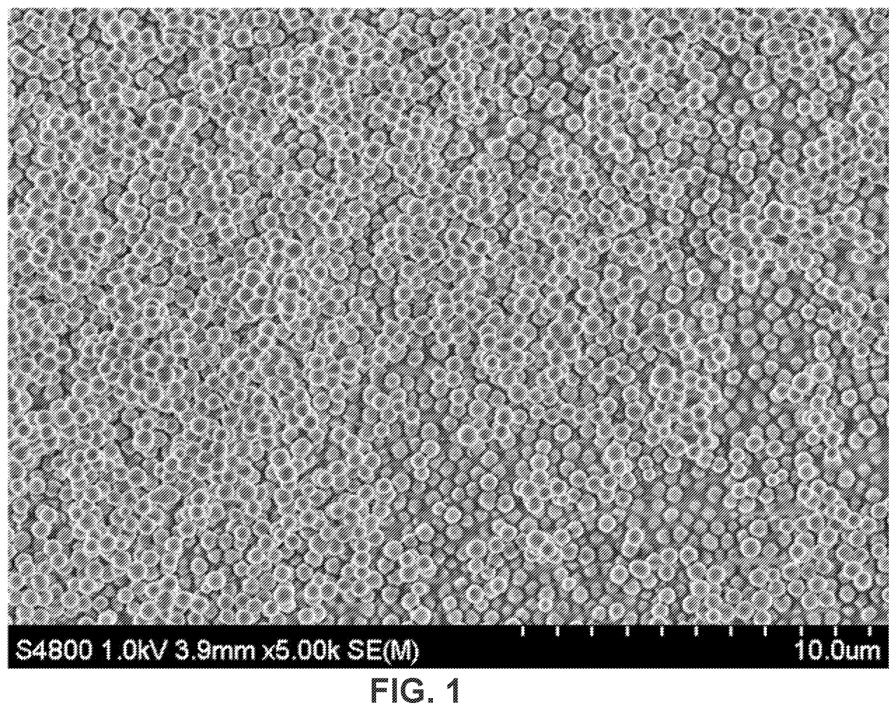

Scanning electron microscopy (SEM) images were collected on a Hitachi Model S-4800 field emission scanning electron microscope. N2 sorption isotherms were measured with a Micromeritics ASAP 2420 analyzer at −196° C. Before the measurements, all samples were degassed at 180° C. in a vacuum for at least 6 hours. FIG. 1 is a Scanning Electron Microscope (SEM) image of a first group of hollow mesoporous silica nanospheres (HMSN-1).

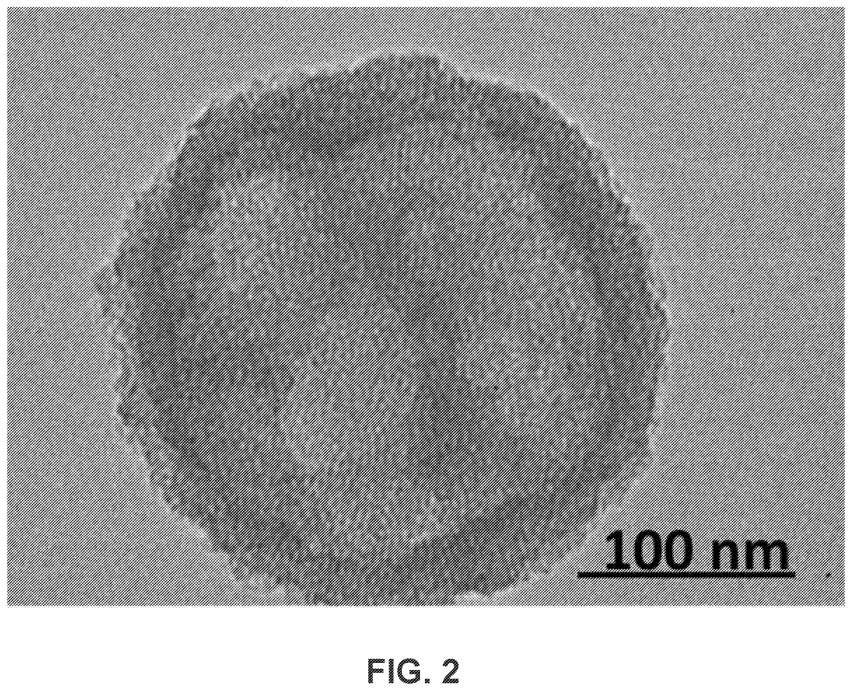

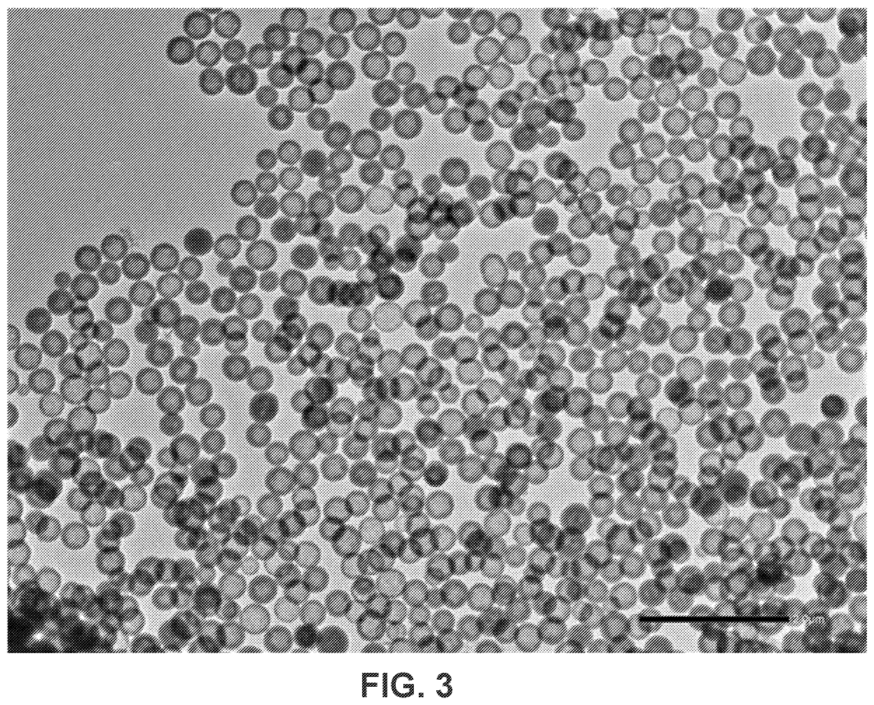

Transmission electron microscopy (TEM) experiments were preformed on a JEOL 1400Plus microscope operated at 120 kV. The ground samples for TEM measurements were suspended in ethanol and supported onto carbon-coated copper grids. FIG. 2 is a Transmission Electron Microscope (TEM) image of HMSN-1. FIG. 3 is a TEM image of a hollow mesoporous silica nanosphere from a second group of hollow mesoporous silica nanospheres (HMSN-2).

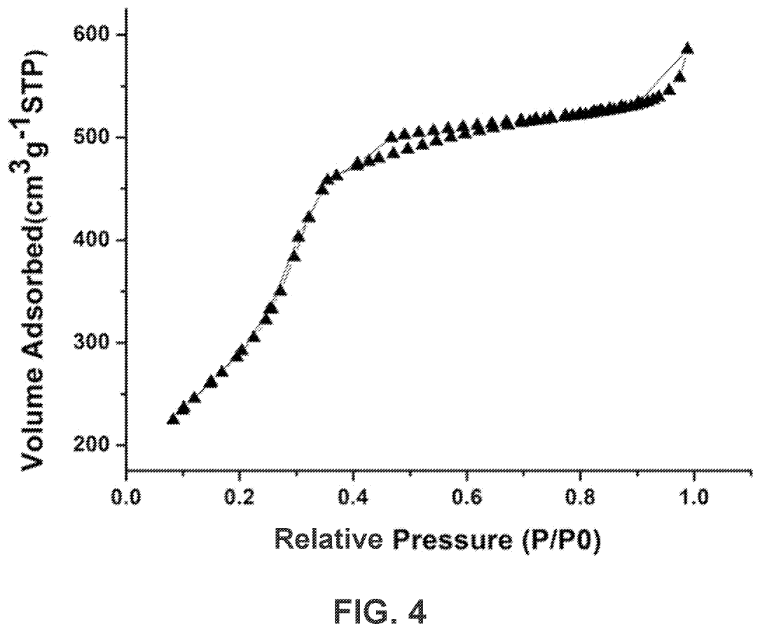

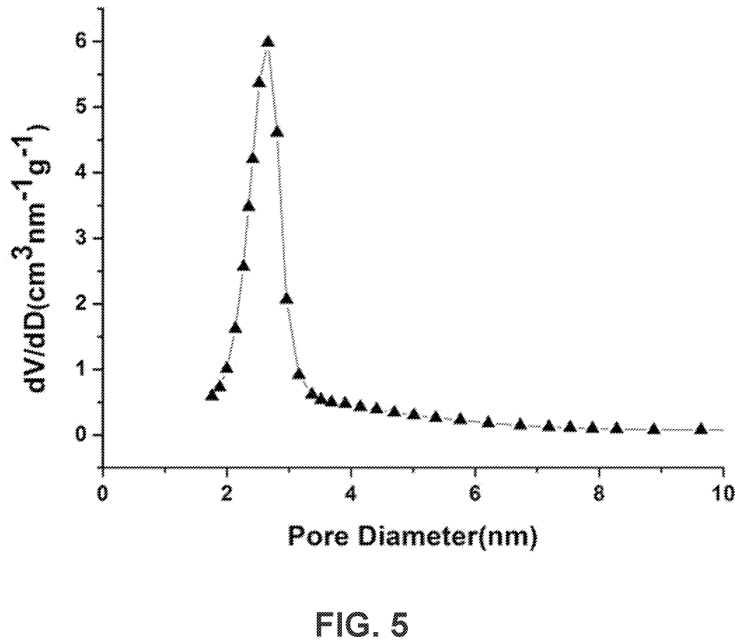

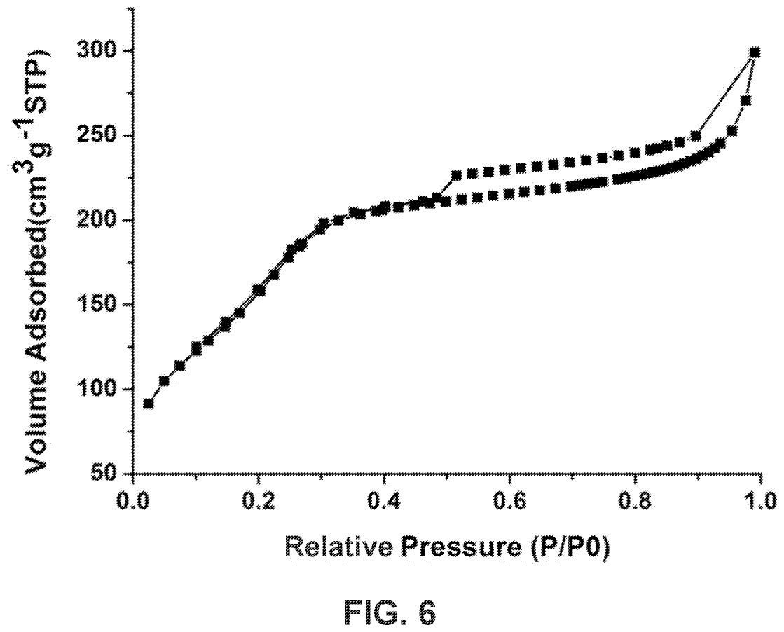

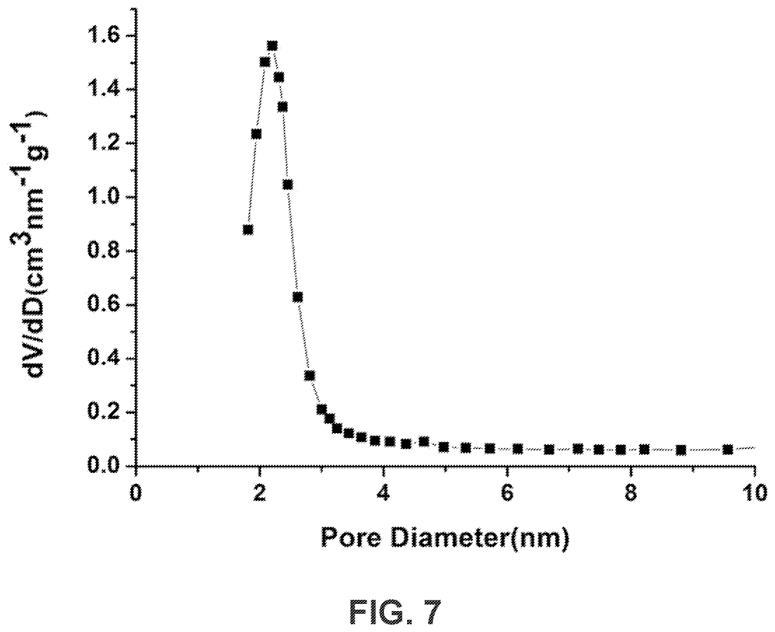

By using the Barrett-Joyner-Halenda (BJH) model, the pore size distributions were derived from the adsorption branches of isotherms. The total pore volumes were calculated based on the adsorbed amounts of nitrogen at a relative pressure of 0.99. The Brunauer-Emmett-Teller (BET) method was utilized to calculate the specific surface areas. FIG. 4 is a plot of nitrogen sorption isotherms for HMSN-1. FIG. 5 is a plot of pore size distribution for HMSN-1. FIG. 6 is a plot of nitrogen sorption isotherms for HMSN-2. FIG. 7 is a plot of pore size distribution for HMSN-2.

HMSN-1 has a particle size of about 250˜300 nm and a mesoporous shell of about 25 nm in thickness. The surface area is ˜960 m²/g and the pore size is about 2.4 nm.

HMSN-2 has particle size of about 350˜400 nm and a mesoporous shell of about 60 nm in thickness, with a hollow cavity about 250 nm in diameter. The surface area is ˜620 m²/g, the pore size is about 2.2 nm.

### Example 2 (Comparative)

To ascertain the SPF of comparative sun care compositions, sunscreen formulations Comparative Batch A, Comparative B, and Comparative Batch C were prepared having the ingredients as listed in TABLES 1 and 2.

TABLE 1

| | | Comparative | Comparative |
|---|---|---|---|
| Phase | Component | Batch A | Batch B |
| Oil | NEO HELIOPAN ™ AV Ethylhexyl Methoxycinnimate | 7.0% | 7.5% |
| Phase | NEO HELIOPAN ™ OS Ethylhexyl Salicylate | 5.0% | 5.0% |
| | NEO HELIOPAN ™ 303 Octocrylene | — | 3.0% |
| | NEO HELIOPAN ™ 357 Butyl Methoxydibenzoylmethane | 1.0% | 2.5% |
| | CRODAMOL ™ GTCC Caprylic/Capric Triglyceride | 7.0% | 10.0% |
| | XIAMETER ™ PMX-245 Silicone Fluid, Cyclopentasiloxane | 5.0% | 5.0% |
| | DOWSIL ™ EL-7040 Hydro Elastomer Blend, Caprylyl | 5.0% | 5.0% |
| | Methicone (and) PEG-12 Dimethicone/PPG-20 Crosspolymer | | |
| | ACULYN ™ SILTOUCH Rheology Modifier, Sodium | 5.0% | 5.0% |
| | Acrylate/Sodium Acryloyldimethyl Taurate Copolymer (and) | | |
| | Dimethicone (and) Trideceth-6 (and) PEG/PPG-18/18 | | |
| | Dimethicone | | |
| | HOSTAPHAT ™ KL 340 Trilaureth-4 Phosphate | 3.0% | 2.0% |
| Aqueous | Water | 58.4% | 51.4% |
| Phase | 1,3-Butylene Glycol | 3.0% | 3.0% |
| | VERSENE ™ Na2 Crystals Chelating Agent, Disodium | 0.1% | 0.1% |
| | ethylenediaminetetraacetate dihydrate | | |
| | EUXYL ™ PE 9010 Phenoxyethanol and Ethylhexylglycerin | 0.5% | 0.5% |

TABLE 2

| | | Comparative |
|---|---|---|
| Phase | Ingredients | Batch C |
| Oil phase | PARSOL ™ MCX Ethylhexyl Methoxycinnamate | 7.0% |
| | PARSOL ™ 1789 Butyl Methoxydibenzoylmethane | 2.0% |
| | PARSOL ™ 5000 4-Methylbenzylidene Camphor | 1.0% |
| | MYRITOL ™ 318 Caprylic/Capric Triglyceride | 5.0% |
| | DOW CORNING ® 556 Phenyl Trimethicone | 2.0% |
| | DOW CORNING ® FZ-3196 Caprylyl Methicone | 2.0% |
| | DOW CORNING FA-4003 DM Dimethicone (and) | 2.0% |
| | Acrylates/Polytrimethylsiloxymethacrylate Copolymer | |
| | Arlacel 165 Glyceryl Stearate (and) PEG-100 Stearate | 0.5% |
| | Cetearyl Alcohol 30/70 | 1.0% |
| | Montanov L C14-22 Alcohol (and) C12-20 Alkyl Glucoside | 3.0% |
| Aqueous | EUXYL ™ PE 9010 Phenoxyethanol and Ethylhexylglycerin | 0.5% |
| phase | WATER | 68.7% |
| | Glycerin | 5.0% |
| | VERSENE ™ Na2 Crystals Chelating Agent, Disodium | 0.1% |
| | ethylenediaminetetraacetate dihydrate | |
| | Xanthan gum | 0.2% |

Amounts are listed by weight percent of the composition. Although “%” is listed in the above table, it is intended to be synonymous with “wt. %”.

The oil phase was prepared by mixing oil phase components and heating to 75° C. to allow the solid ingredients to melt and form a homogeneous mixture.

The aqueous phase (not including the preservatives) was prepared by mixing the aqueous phase components together and heating to 75° C.

The oil phase was mixed into the aqueous phase with agitation. After complete mixing, the mixture was cooled to 40° C. while maintaining agitation. Next, the preservative EUXYL® PE 9010 was added, and the mixture was cooled to room temperature.

### Example 3

To ascertain the SPF boosting efficiency of hollow mesoporous silica nanospheres, HMSN-1 and HMSN-2 substantially as described in Example 1 are incorporated into sun care compositions (e.g., sunscreen formulations) were prepared having the ingredients as listed in TABLES 3 and 4.

TABLE 3

| Phase | Component | Batch 1 | Batch 2 |
|---|---|---|---|
| Oil Phase | NEO HELIOPAN ™ AV Ethylhexyl Methoxycinnimate | 7.0% | 7.5% |
| | NEO HELIOPAN ™ OS Ethylhexyl Salicylate | 5.0% | 5.0% |
| | NEO HELIOPAN ™ 303 Octocrylene | — | 3.0% |
| | NEO HELIOPAN ™ 357 Butyl Methoxydibenzoylmethane | 1.0% | 2.5% |
| | CRODAMOL ™ GTCC Caprylic/Capric Triglyceride | 7.0% | 10.0% |
| | XIAMETER ™ PMX-245 Silicone Fluid, Cyclopentasiloxane | 5.0% | 5.0% |
| | DOWSIL ™ EL-7040 Hydro Elastomer Blend, Caprylyl | 5.0% | 5.0% |
| | Methicone (and) PEG-12 Dimethicone/PPG-20 Crosspolymer | | |
| | ACULYN ™ SILTOUCH Rheology Modifier, Sodium | 5.0% | 5.0% |
| | Acrylate/Sodium Acryloyldimethyl Taurate Copolymer (and) | | |
| | Dimethicone (and) Trideceth-6 (and) PEG/PPG-18/18 | | |
| | Dimethicone | | |
| | HOSTAPHAT ™ KL 340 Trilaureth-4 Phosphate | 3.0% | 2.0% |
| Aqueous | Water | 55.1% | 46.4% |
| Phase | 1,3-Butylene Glycol | 3.0% | 3.0% |
| | VERSENE ™ Na2 Crystals Chelating Agent, Disodium | 0.1% | 0.1% |
| | ethylenediaminetetraacetate dihydrate | | |
| | Hollow Mesoporous Silica Nanosphere Batch 1 (HMSN-1) | 3.3% | 5.0% |
| | EUXYL ™ PE 9010 Phenoxyethanol and Ethylhexylglycerin | 0.5% | 0.5% |

TABLE 4

| Phase | Components | Batch 3 | Batch 4 |
|---|---|---|---|
| Oil phase | PARSOL ™ MCX Ethylhexyl Methoxycinnamate | 7.0% | 7.0% |
| | PARSOL ™ 1789 Butyl Methoxydibenzoylmethane | 2.0% | 2.0% |
| | PARSOL ™ 5000 4-Methylbenzylidene Camphor | 1.0% | 1.0% |
| | MYRITOL ™ 318 Caprylic/Capric Triglyceride | 5.0% | 5.0% |
| | DOW CORNING ® 556 Phenyl Trimethicone | 2.0% | 2.0% |
| | DOW CORNING ® FZ-3196 Caprylyl Methicone | 2.0% | 2.0% |
| | DOW CORNING FA-4003 DM Dimethicone (and) | 2.0% | 2.0% |
| | Acrylates/Polytrimethylsiloxymethacrylate Copolymer | | |
| | Arlacel 165 Glyceryl Stearate (and) PEG-100 Stearate | 0.5% | 0.5% |
| | Cetearyl Alcohol 30/70 | 1.0% | 1.0% |
| | Montanov L C14-22 Alcohol (and) C12-20 Alkyl Glucoside | 3.0% | 3.0% |
| Aqueous | EUXYL ™ PE 9010 Phenoxyethanol and Ethylhexylglycerin | 0.5% | 0.5% |
| phase | WATER | 63.7% | 63.7% |
| | Glycerin | 5.0% | 5.0% |
| | VERSENE ™ Na2 Crystals Chelating Agent, Disodium | 0.1% | 0.1% |
| | ethylenediaminetetraacetate dihydrate | | |
| | Xanthan gum | 0.2% | 0.2% |
| | Hollow Mesoporous Silica Nanosphere Batch 3 (HMSN-1) | 5.0% | — |
| | Hollow Mesoporous Silica Nanosphere Batch 4 (HMSN-2) | — | 5.0% |

Amounts are listed by weight percent of the composition. Although “%” is listed in the above table, it is intended to be synonymous with “wt. %”.

The oil phase was prepared by mixing oil phase components and heating to 75° C. to allow the solid ingredients to melt and form a homogeneous mixture.

The aqueous phase (not including the preservatives) was prepared by dispersing, in a separate vessel, the recited HMSN powder in water with homogenization at 8000 rpm, and then mixing all the aqueous phase components together and heating to 75° C.

The oil phase was mixed into the aqueous phase with agitation. After complete mixing, the mixture was cooled to 40° C. while maintaining agitation. Next, the preservative EUXYL® PE 9010 was added, and the mixture was cooled to room temperature.

### Example 4

The respective sun care compositions from Examples 2 and 3 were each coated on a 5 cm×5 cm PMMA plate at level of 1.2-1.3 mg/cm², then dried at room temperature for 15 min before measurement. The sun protection factor (SPF) was measured using a PerkinElmer Lambda 950 Ultraviolet Transmittance Analyzer with an integrating spheres and SPF Operating Software. The UV absorbance of a sample over UV radiation wavelengths (290-400 nm for each sample) was measured, and SPF value was calculated based on this UV absorbance spectrum.

Using the weight of the dry film, and the solids content of the layer, the density of the original wet layer immediately after deposition can be calculated. Using this information, the SPF can be calculated by the following Equation (5):

$$S P F = \frac{{\int }_{290 nm}^{400 nm} E ( λ ) S ( λ ) \partial λ}{{\int }_{290 nm}^{400 nm} E ( λ ) S ( λ ) {10}^{( - A ( λ ) )} \partial λ} \qquad ( 5 )$$

Where E(λ)=spectral irradiance of the Standard Sun Spectrum; S(λ)=erythemal action spectrum at wavelength λ; and A(λ)=corrected spectral absorbance at wavelength λ (a correction factor is calculated to extrapolate the data to establish what the absorbance would be at a wet layer density of 2.0 mg/cm2 (using the original wet layer immediately after deposition)).

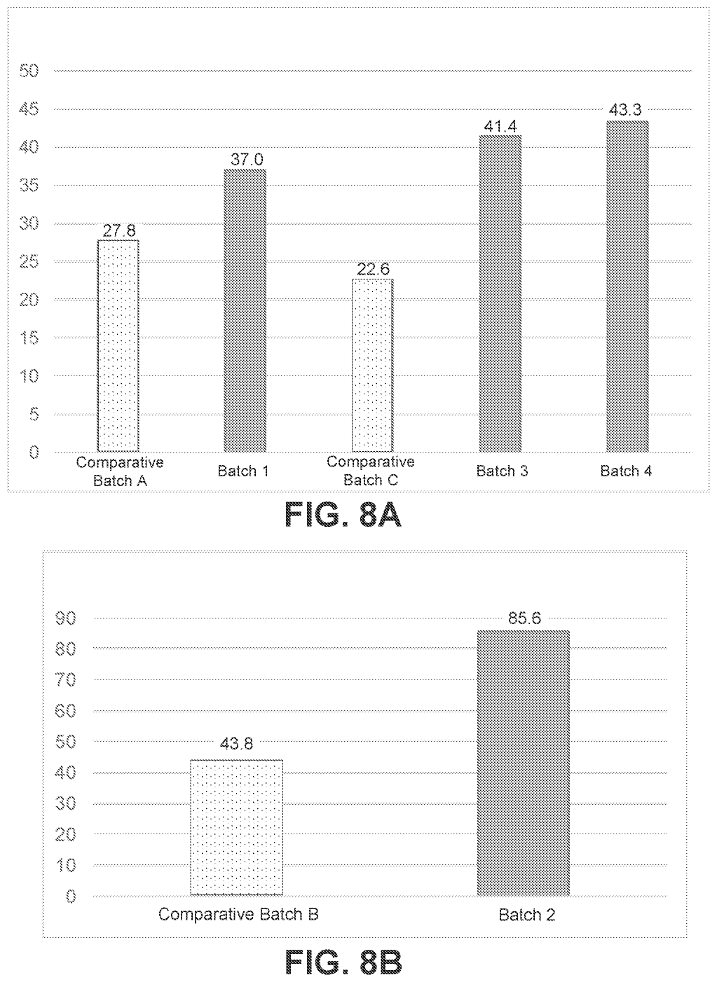

FIG. 8A is a diagram of SPF measurements for comparative sun care formulations (Comparative Batch A and Comparative Batch C from Example 2), and sun care formulations incorporating HMSN-1 (Batch 1 and Batch 3 from Example 3), and a sun care formulation incorporating HMSN-2 (Batch 4 from Example 3).

FIG. 8B is a diagram of SPF measurements for a comparative sun care formulation (Comparative Batch B) and a sun care formulation incorporating HMSN-1 (Batch 2 from Example 3).

With 3.3 wt. % of HMSNs, the SPF demonstrated about a 33% increase from 27.8 (Comparative Batch A) to 37.0 (Batch 1 (containing HMSN-1)). With 5.0 wt. % of HMSNs, the SPF demonstrated about a 95% increase from 43.8 (Comparative Batch B) to 85.6 (Batch 2 (containing HMSN-1)). Moreover, the SPF increased 83%-92% from 22.6 (Comparative Batch C) to 41.4 (Batch 3 (containing HMSN-1)) and 43.3 (Batch 4 (containing HMSN-2)). The introduction of HMSNs in sunscreen compositions significantly increased the SPF as compared to the comparative sunscreens (e.g., where HMSNs were not present). Accordingly, hollow mesoporous silica nanospheres act as SPF boosters in sun care compositions.

It is understood that this disclosure is not limited to the embodiments specifically disclosed and exemplified herein. Various modifications of the invention will be apparent to those skilled in the art. Such changes and modifications may be made without departing from the scope of the appended claims. Moreover, each recited range includes all combinations and sub-combinations of ranges, as well as specific numerals contained therein.

1. A sun care composition, comprising:
at least one sunscreen active; and
hollow mesoporous silica nanospheres, wherein the mesoporous silica nanospheres have a spherical hollow cavity with a diameter of between 100 nm and 300 nm.

2. The sun care composition of claim 1, wherein the at least one sunscreen active is a mixture of ethylhexyl methoxycinnamate, ethylhexyl salicylate, and butyl methoxydibenzoylmethane.

3. The sun care composition of claim 1, wherein the hollow mesoporous silica nanospheres are about 3.3% by weight of the sun care composition.

4. The sun care composition of claim 2, further comprising 2-ethylhexyl-2-cyano-3,3 diphenylacrylate.

5. The sun care composition of claim 1, wherein the hollow mesoporous silica nanospheres are about 5% by weight of the sun care composition.

6. The sun care composition of claim 1, wherein the hollow mesoporous silica nanospheres have a particle size from between about 150 nm and about 400 nm.

7. The sun care composition of claim 1, wherein the hollow mesoporous silica nanospheres have a surface area of between about 600 m²/g nm and 1200 m²/g.

8. The sun care composition of claim 1, wherein the hollow mesoporous silica nanospheres have a pore size of between about 2.0 nm and 4.0 nm.

9. The sun care composition of claim 1, further comprising at least one of a cosmetically acceptable emollient, humectant, vitamin, moisturizer, conditioner, oil, silicone, suspending agent, surfactant, emulsifier, preservative, rheology modifier, pH adjustor, reducing agent, anti-oxidant, and/or foaming or de-foaming agent.

10. The sun care composition of claim 2, wherein a sun protection factor (SPF) of the sun care composition is more than 25% higher than a comparative composition without the hollow mesoporous silica nanospheres.

11. The sun care composition of claim 2, wherein the hollow mesoporous silica nanospheres are about 3.3% by weight of the sun care composition.

12. The sun care composition of claim 4, wherein the hollow mesoporous silica nanospheres are about 5% by weight of the sun care composition.

13. The sun care composition of claim 4, wherein a sun protection factor (SPF) of the sun care composition is more than 25% higher than a comparative composition without the hollow mesoporous silica nanospheres.

14. A sun care composition, comprising:
a sunscreen active mixture comprising ethylhexyl methoxycinnamate, ethylhexyl salicylate, and butyl methoxydibenzoylmethane; and
hollow mesoporous silica nanospheres, wherein the mesoporous silica nanospheres have a spherical hollow cavity with a diameter of between 100 nm and 300 nm, and wherein the hollow mesoporous silica nanospheres comprise greater than about 2% and less than about 7% by weight of the sun care composition.

15. The sun care composition of claim 14, wherein the hollow mesoporous silica nanospheres are about 3.3% by weight of the sun care composition.

16. The sun care composition of claim 14, wherein a sun protection factor (SPF) of the sun care composition is more than 25% higher than a comparative composition without the hollow mesoporous silica nanospheres.

17. The sun care composition of claim 14, wherein the at least one sunscreen active mixture further comprises 2-ethylhexyl-2-cyano-3,3 diphenylacrylate.

18. The sun care composition of claim 17, wherein the hollow mesoporous silica nanospheres are about 5% by weight of the sun care composition.

19. The sun care composition of claim 17, wherein a sun protection factor (SPF) of the sun care composition is more than 25% higher than a comparative composition without the hollow mesoporous silica nanospheres.

20. The sun care composition of claim 14, wherein the hollow mesoporous silica nanospheres have:
a particle size from between about 150 nm and about 400 nm;
a surface area of between about 600 m²/g nm and 1200 m²/g; and
a pore size of between about 2.0 nm and 4.0 nm.
